(19) United States
(12) Patent Application Publication
(10) Pub. No.: US 2024/0315868 A1
(43) Pub. Date: Sep. 26, 2024

(54) ANTI-SNORING DEVICE

(71) Applicant: YUNHYUNG SON, LEONIA, NJ (US)

(72) Inventor: YUNHYUNG SON, LEONIA, NJ (US)

(21) Appl. No.: 18/125,076

(22) Filed: Mar. 22, 2023

(51) Int. Cl. A61F 5/56; A61F 13/12

(52) U.S. Cl. CPC A61F 5/56; A61F 13/122

(57) ABSTRACT

The present invention is an anti-snoring device that includes a non-woven fabric layer, a plurality of spaced apart apertures formed to extent through the non-woven fabric layer, an adhesive layer, a plurality of spaced apart apertures formed to extent through the adhesive layer, and a removable backing. The non-woven fabric layer is configured to fit onto a portion of a user's upper and lower lips. The adhesive layer is configured to facilitate removably attaching the non-woven fabric layer to the portion of the user's upper and lower lips. The removable backing is configured to cover the adhesive layer.

### TECHNICAL FIELD & BACKGROUND

The present invention is generally related to an anti-snoring device, and in particular, to an improved anti-snoring device.

Many people lose sleep due to snoring. Typically, snoring occurs when your tongue and tissues in your mouth and throat become too relaxed. Air passing through creates vibrations in the tissue, producing the snoring sound.

Since snoring can be highly disruptive, snorers often use mouthpieces or mouthguards to reduce snoring.

However, some people find such mouthpieces or mouthguards uncomfortable and even painful at times. In fact, such mouthpieces or mouthguards could carry more potential side effects, such as jaw pain and stiffness, drooling, or dry mouth.

Moreover, people with braces, dentures, dental implants, or broken teeth would not be able to use such mouthpieces or mouthguards.

Therefore, it would be desirable to provide an anti-snoring device that not only could be used for any users (including users with braces, dentures, dental implants, or broken teeth) but also could reduce snoring without such side effects or pain.

### SUMMARY OF THE INVENTION

The present invention is directed to an anti-snoring device that overcomes the above-mentioned disadvantages of the prior art.

In one aspect, the present invention provides an anti-snoring device having a non-woven fabric layer, a plurality of spaced apart apertures formed to extent through the non-woven fabric layer, an adhesive layer, a plurality of spaced apart apertures formed to extent through the adhesive layer, and a removable backing. The non-woven fabric layer has a top surface, a corresponding bottom surface, and a marginal edge portion integral with and surrounding the top surface and the bottom surface. The non-woven fabric layer is configured to fit onto a portion of a user's upper and lower lips. The plurality of spaced apart apertures formed to extent through the non-woven fabric layer provides air flow between the top surface and the bottom surface of the non-woven fabric layer. The adhesive layer has an upper surface and a corresponding lower surface. The upper surface of the adhesive layer is attached to the bottom surface of the non-woven fabric layer. The adhesive layer is configured to facilitate removably attaching the non-woven fabric layer to the portion of the user's upper and lower lips. The plurality of spaced apart apertures formed to extent through the adhesive layer provides air flow between the upper surface and the lower surface of the adhesive layer. The removable backing is configured to cover the non-woven fabric layer. The removable backing is removably attached to the lower surface of the adhesive layer.

In another aspect, the present invention provides an anti-snoring device having a non-woven fabric layer, a plurality of spaced apart apertures formed to extent through the non-woven fabric layer, an adhesive layer, a plurality of spaced apart apertures formed to extent through the adhesive layer, and a removable backing. The non-woven fabric layer has a top surface, a corresponding bottom surface, and a marginal edge portion integral with and surrounding the top surface and the bottom surface. The non-woven fabric layer is configured to fit onto a portion of a user's upper and lower lips. The plurality of spaced apart apertures formed to extent through the non-woven fabric layer provides air flow between the top surface and the bottom surface of the non-woven fabric layer. The adhesive layer has an upper surface and a corresponding lower surface. The upper surface of the adhesive layer is attached to the bottom surface of the non-woven fabric layer. The adhesive layer is configured to facilitate removably attaching the non-woven fabric layer to the portion of the user's upper and lower lips. The plurality of spaced apart apertures formed to extent through the adhesive layer provides air flow between the upper surface and the lower surface of the adhesive layer. The removable backing is configured to cover the adhesive layer. The removable backing is removably attached to the lower surface of the adhesive layer. The removable backing has an identical width and length of the adhesive layer.

In a further aspect, the present invention provides an anti-snoring device having a non-woven fabric layer, a plurality of spaced apart apertures formed to extent through the non-woven fabric layer, an adhesive layer, a plurality of spaced apart apertures formed to extent through the adhesive layer, and a removable backing. The non-woven fabric layer has a top surface, a corresponding bottom surface, and a marginal edge portion integral with and surrounding the top surface and the bottom surface. The non-woven fabric layer is configured to fit onto a portion of a user's upper and lower lips. The plurality of spaced apart apertures formed to extent through the non-woven fabric layer provides air flow between the top surface and the bottom surface of the non-woven fabric layer. The adhesive layer has an upper surface and a corresponding lower surface. The upper surface of the adhesive layer is attached to the bottom surface of the non-woven fabric layer. The adhesive layer is configured to facilitate removably attaching the non-woven fabric layer to the portion of the user's upper and lower lips. The plurality of spaced apart apertures formed to extent through the adhesive layer provides air flow between the upper surface and the lower surface of the adhesive layer. The removable backing is configured to cover the non-woven fabric layer and the adhesive layer. The removable backing is removably attached to the lower surface of the adhesive layer. The non-woven fabric layer has a perimeter and includes a flexible member encasing the perimeter. The adhesive layer has an identical width and length of the non-woven fabric layer.

### BRIEF DESCRIPTION OF THE DRAWINGS

The present invention will be described by way of exemplary embodiments, but not limitations, illustrated in the accompanying drawing in which like references denote similar elements, and in which:

FIG. 1 illustrates a perspective view of an anti-snoring device, in accordance with one embodiment of the present invention.

FIG. 2 illustrates an exploded perspective view of an anti-snoring device, in accordance with one embodiment of the present invention.

FIG. 3 illustrates a front view of an anti-snoring device, in accordance with one embodiment of the present invention.

FIG. 4 illustrates a back view of an anti-snoring device, in accordance with one embodiment of the present invention.

FIG. 5 illustrates an exploded back view of an anti-snoring device, in accordance with one embodiment of the present invention.

FIG. 6 illustrates a side view of an anti-snoring device, in accordance with one embodiment of the present invention.

FIG. 7 illustrates a front view of an anti-snoring device being held by a user, in accordance with one embodiment of the present invention.

FIG. 8 illustrates a front view of an anti-snoring device being worn by a user, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments. Various operations will be described as multiple discrete operations, in turn, in a manner that is most helpful in understanding the present invention. However, the order of description should not be construed as to imply that these operations are necessarily order dependent. In particular, these operations need not be performed in the order of presentation. The phrase “in one embodiment” is used repeatedly. The phrase generally does not refer to the same embodiment, however, it may. The terms “comprising”, “having” and “including” are synonymous, unless the context dictates otherwise.

This present invention provides a breathable anti-snoring device that not only could be used for any users (including users with braces, dentures, dental implants, or broken teeth) but also could reduce snoring without the side effects or pain that mouthpieces or mouthguards may cause.

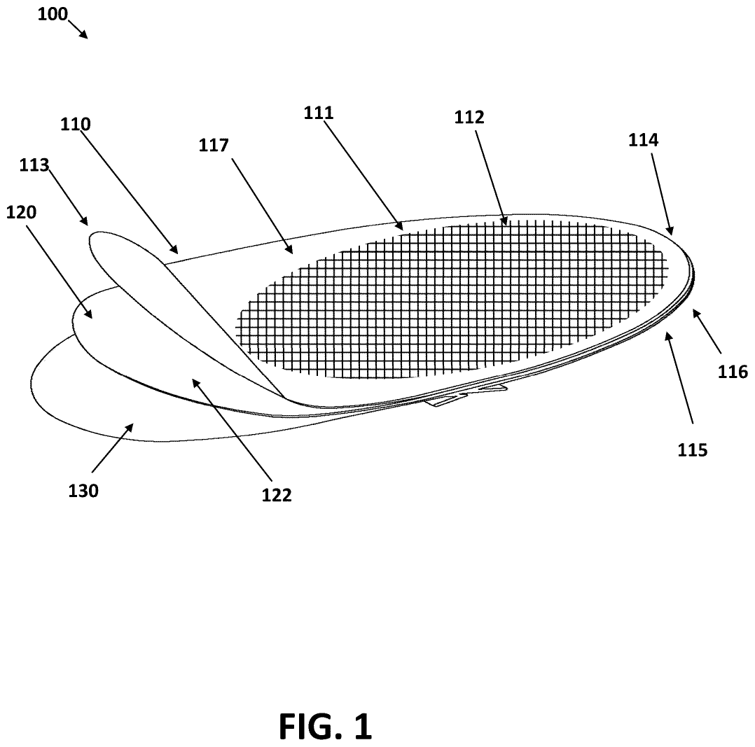

FIG. 1 illustrates a perspective view of an anti-snoring device 100, in accordance with one embodiment of the present invention.

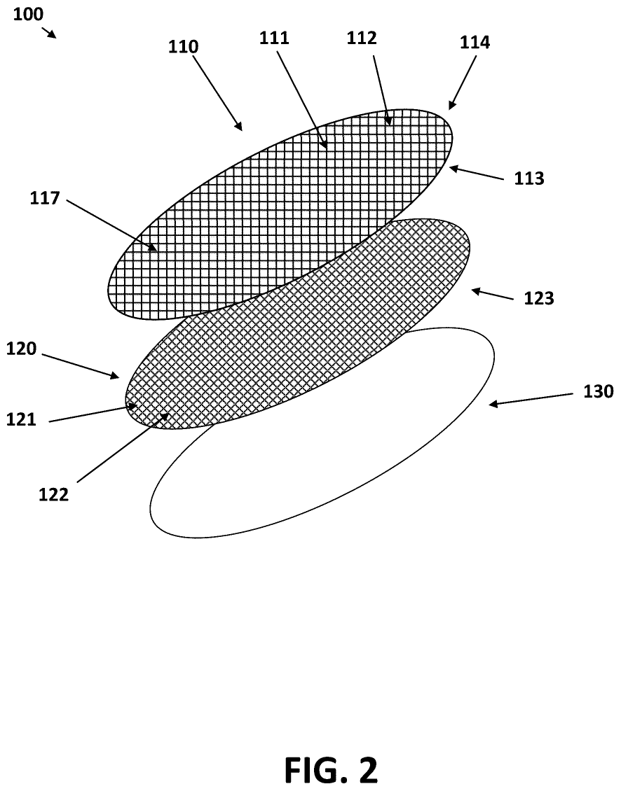

FIG. 2 illustrates an exploded perspective view of an anti-snoring device 100, in accordance with one embodiment of the present invention.

Referring to FIGS. 1 and 2, an anti-snoring 100 device includes a non-woven fabric layer 110, a plurality of spaced apart apertures 111 formed to extent through the non-woven fabric layer 110, an adhesive layer 120, a plurality of spaced apart apertures 121 formed to extent through the adhesive layer 120, and a removable backing 130.

The non-woven fabric layer 110 has a top surface 112, a corresponding bottom surface 113, and a marginal edge portion 114 integral with and surrounding the top surface 112 and the bottom surface 113. The non-woven fabric layer 110 is configured to fit onto a portion of a user's upper and lower lips. The plurality of spaced apart apertures 111 formed to extent through the non-woven fabric layer 110 provides air flow between the top surface 112 and the bottom surface 113 of the non-woven fabric layer 110.

Preferably, the non-woven fabric layer 110 may be formed substantially or entirely from viscose rayon. Viscose rayon has the advantages of being breathable and moisture absorbent.

Most importantly, the non-woven fabric made from viscose rayon is a very light fabric that provides excellent air flow when it is on a user's lips. As a result, such non-woven fabric made from viscose rayon makes the present invention the most safe and efficient anti-snoring device that could provide enough air flow to the user through the lips during sleep.

This is an unexpected result found by the applicant and serves as a fundamental basis of the present invention.

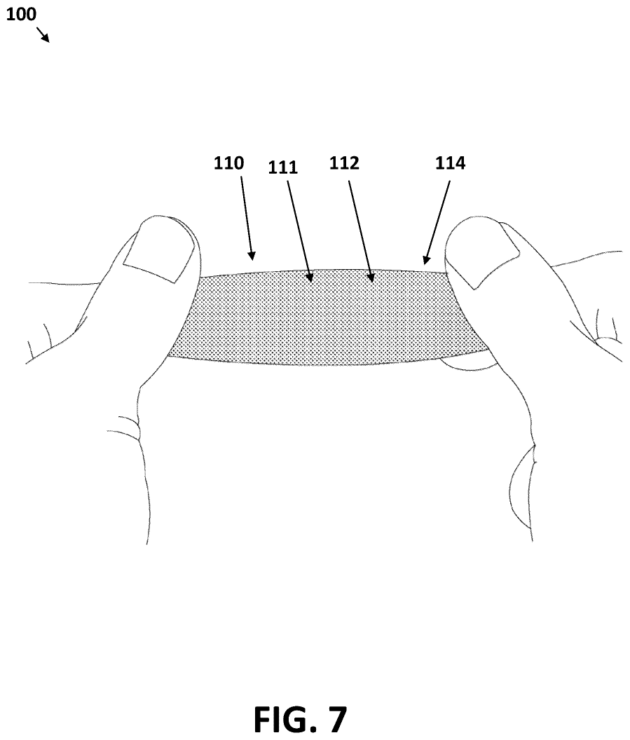

FIG. 7 illustrates a front view of an anti-snoring device being held by a user, in accordance with one embodiment of the present invention.

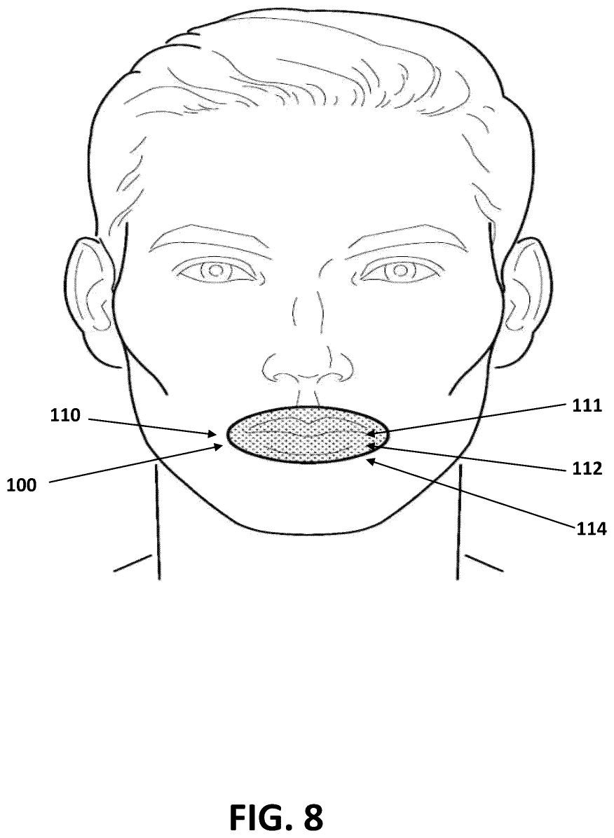

FIG. 8 illustrates a front view of an anti-snoring device being worn by a user, in accordance with one embodiment of the present invention.

In an exemplary embodiment, and as illustrated in FIGS. 7 and 8, the non-woven fabric layer 110 may have thicknesses in the range of 0.45 mm to 0.55 mm. The non-woven fabric layer may have a weight in the range 2.8 to 3.5 ounces per square yard. The thicknesses and weight were found by the applicant to provide the most acceptable stretch, comfort and strength.

Preferably, the non-woven fabric layer 110 may have a width in the range of 34 mm to 36 mm. The non-woven fabric layer 110 may have a length in the range of 79 mm to 81 mm. The height and length were found by the applicant to provide the most comfort and suitable size for the most users' lips.

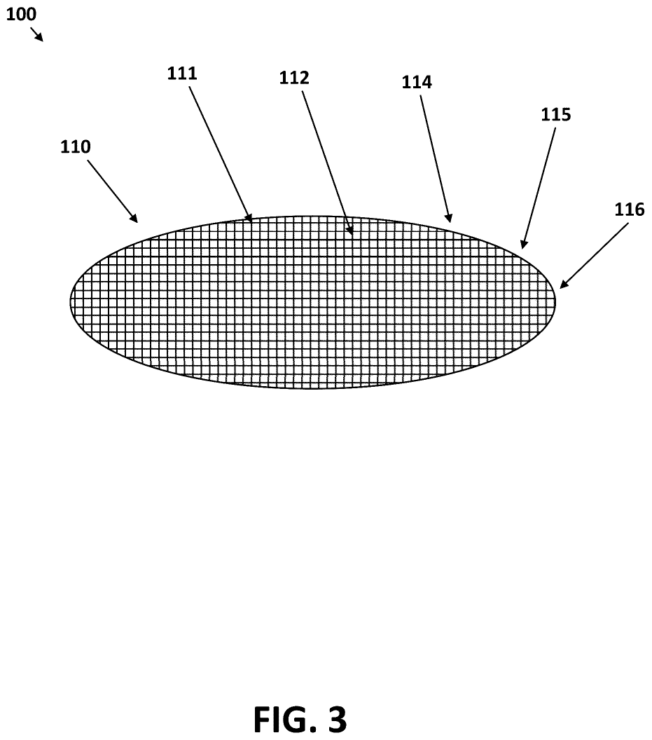

FIG. 3 illustrates a front view of an anti-snoring device, in accordance with one embodiment of the present invention.

Referring to FIG. 3, the non-woven fabric layer 110 may have a perimeter 115. The perimeter may include a flexible member 116 encasing the perimeter 115.

Preferably, the flexible member 116 may be made of a plastic, silicone, rubber, non-woven fabric, or woven fabric.

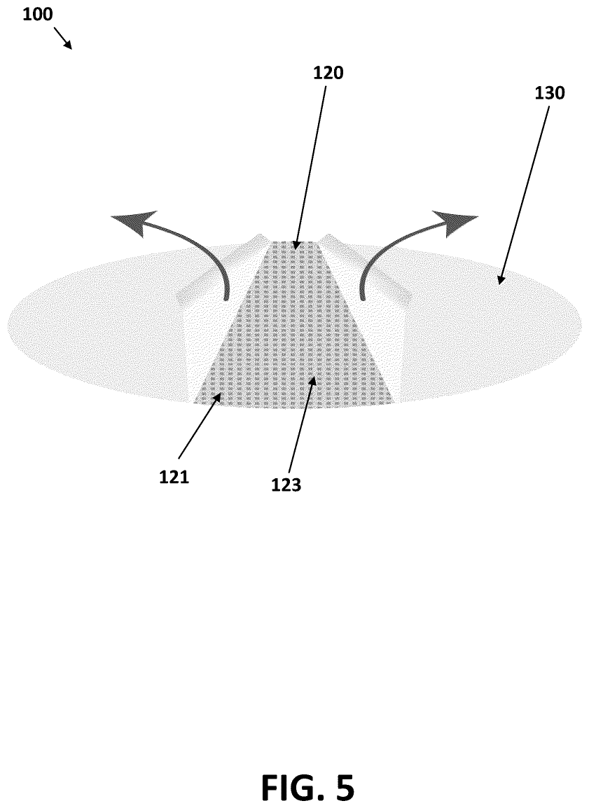

FIG. 5 illustrates an exploded back view of an anti-snoring device, in accordance with one embodiment of the present invention.

Referring to FIGS. 2, 5 and 8, the adhesive layer 120 has an upper surface 122 and a corresponding lower surface 123. The upper surface 122 of the adhesive layer 120 is attached to the bottom surface 113 of the non-woven fabric layer 110. The adhesive layer 120 is configured to facilitate removably attaching the non-woven fabric layer 110 to the portion of the user's upper and lower lips. The plurality of spaced apart apertures 121 formed to extent through the adhesive layer 120 provides air flow between the upper surface 122 and the lower surface 123 of the adhesive layer 120.

Preferably, the adhesive layer 120 may be formed substantially or entirely from acrylate copolymer. The acrylate copolymer showed significant improvement in shine, ease of application, nourishing skin feel and comfort.

In an exemplary embodiment, the adhesive layer 120 may have an identical width and length of the non-woven fabric layer 110. The adhesive layer 120 may have a pattern of stripes.

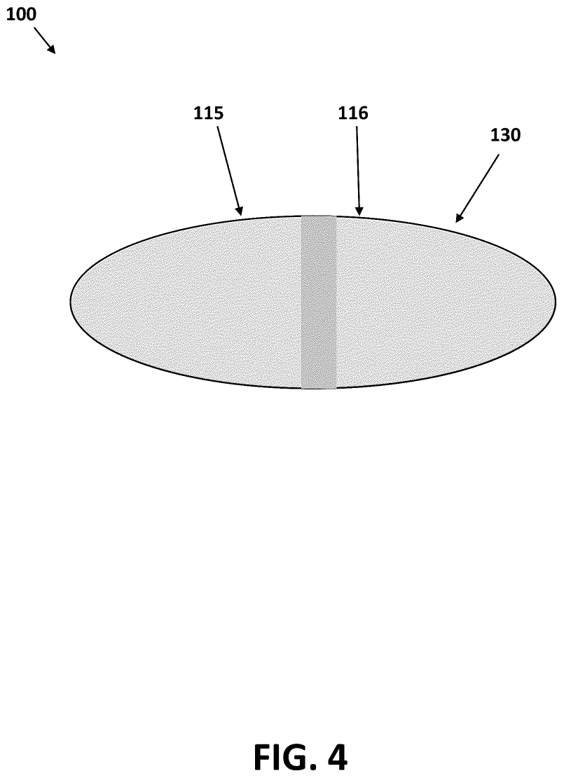

FIG. 4 illustrates a back view of an anti-snoring device, in accordance with one embodiment of the present invention.

Referring to FIGS. 4 and 5, the removable backing 130 may be configured to cover either the non-woven fabric 110 or the adhesive layer 120. The removable backing 130 may also be configured to cover both the non-woven fabric layer 110 and the adhesive layer 120. The removable backing 130 is removably attached to the lower surface 123 of the adhesive layer 120.

Preferably, the removable backing 130 may be one or more of: a cotton backing, a biodegradable plastic material backing, a paper backing. The removable backing 130 may have an identical width and length of the non-woven fabric layer 110 or the adhesive layer 120.

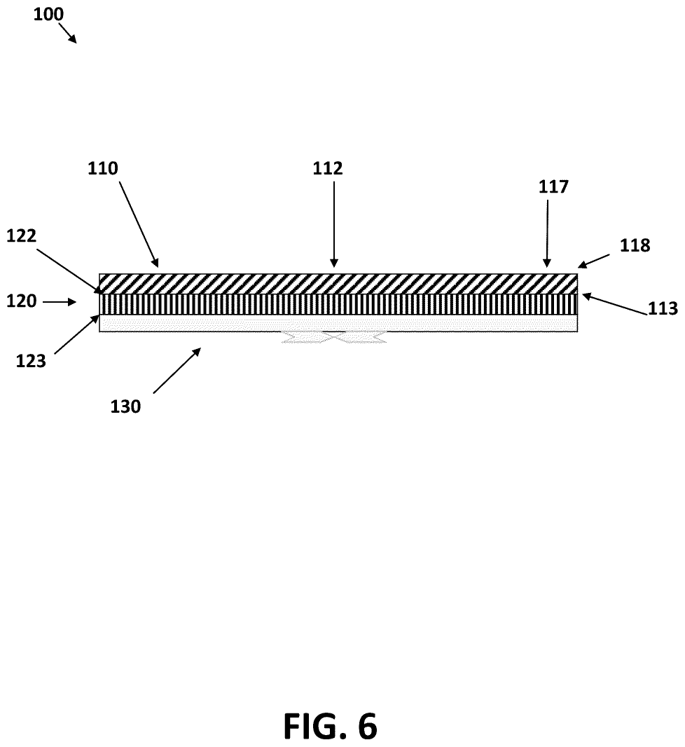

FIG. 6 illustrates a side view of an anti-snoring device, in accordance with one embodiment of the present invention.

Referring to FIG. 6, a laminate 117 may be attached to the top surface 112 of the non-woven fabric layer 110. The laminate 117 may comprise a breathable film 118. The breathable film 118 may be one or more of: a breathable microporous film, a monolithic film.

In an exemplary embodiment, breathability and liquid barrier properties may be obtained by the use of the breathable film 118. Generally, the breathable film 118 is impermeable to liquid water and to bacteria and as such forms a very effective shield which protects a user from sources of infection external to the skin. The vapor permeability of the breathable film 118 can also provide a sufficient rate of air flow through the film to allow the skin to breathe.

Thus, the present invention disclosed herein provides a breathable anti-snoring device that could be used for any users (including users with braces, dentures, dental implants, or broken teeth) without the side effects of mouthpieces or mouthguards by implementing such non-woven fabric layer, adhesive layer, plurality of spaced apart apertures formed to extent through the adhesive and the adhesive layer, and removable backing.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. An anti-snoring device, comprising:
a non-woven fabric layer having a top surface, a corresponding bottom surface, and a marginal edge portion integral with and surrounding the top surface and the bottom surface, wherein the non-woven fabric layer is configured to fit onto a portion of a user's upper and lower lips;
a plurality of spaced apart apertures formed to extent through the non-woven fabric layer to provide air flow between the top surface and the bottom surface of the non-woven fabric layer;
an adhesive layer having an upper surface and a corresponding lower surface, the upper surface of the adhesive layer being attached to the bottom surface of the non-woven fabric layer, wherein the adhesive layer is configured to facilitate removably attaching the non-woven fabric layer to the portion of the user's upper and lower lips;
a plurality of spaced apart apertures formed to extent through the adhesive layer to provide air flow between the upper surface and the lower surface of the adhesive layer; and
a removable backing being configured to cover the non-woven fabric layer, wherein the removable backing is removably attached to the lower surface of the adhesive layer.

2. The anti-snoring device according to claim 1, wherein the non-woven fabric layer is formed from viscose rayon.

3. The anti-snoring device according to claim 1, wherein the adhesive layer is formed from acrylate copolymer.

4. The anti-snoring device according to claim 1, wherein the removable backing is one or more of: a cotton backing, a biodegradable plastic material backing, a paper backing.

5. The anti-snoring device to claim 1, wherein a laminate is attached to the top surface of the non-woven fabric layer, the laminate comprising a breathable film.

6. The anti-snoring device to claim 5, wherein the breathable film is one or more of: a microporous film, a monolithic film.

7. The anti-snoring device to claim 1, wherein the removable backing has an identical width and length of the non-woven fabric layer.

8. An anti-snoring device, comprising:
a non-woven fabric layer having a top surface, a corresponding bottom surface, and a marginal edge portion integral with and surrounding the top surface and the bottom surface, wherein the non-woven fabric layer is configured to fit onto a portion of a user's upper and lower lips;
a plurality of spaced apart apertures formed to extent through the non-woven fabric layer to provide air flow between the top surface and the bottom surface of the non-woven fabric layer;
an adhesive layer having an upper surface and a corresponding lower surface, the upper surface of the adhesive layer being attached to the bottom surface of the non-woven fabric layer, wherein the adhesive layer is configured to facilitate removably attaching the non-woven fabric layer to the portion of the user's upper and lower lips;
a plurality of spaced apart apertures formed to extent through the adhesive layer to provide air flow between the upper surface and the lower surface of the adhesive layer;
a removable backing being configured to cover the adhesive layer, wherein the removable backing is removably attached to the lower surface of the adhesive layer; and
wherein the removable backing has an identical width and length of the adhesive layer.

9. The anti-snoring device according to claim 8, wherein the non-woven fabric layer is formed from viscose rayon.

10. The anti-snoring device according to claim 8, wherein the adhesive layer is formed from acrylate copolymer.

11. The anti-snoring device according to claim 8, wherein the removable backing is one or more of: a cotton backing, a biodegradable plastic material backing, a paper backing.

12. The anti-snoring device according to claim 8, wherein a laminate is attached to the top surface of the non-woven fabric layer, the laminate comprising a breathable film.

13. The anti-snoring device according to claim 12, wherein the breathable film is one or more of: a microporous film, a monolithic film.

14. The anti-snoring device to claim 8, wherein the removable backing has an identical width and length of the non-woven fabric layer.

15. An anti-snoring device, comprising:
a non-woven fabric layer having a top surface, a corresponding bottom surface, and a marginal edge portion integral with and surrounding the top surface and the bottom surface, wherein the non-woven fabric layer is configured to fit onto a portion of a user's upper and lower lips;
a plurality of spaced apart apertures formed to extent through the non-woven fabric layer to provide air flow between the top surface and the bottom surface of the non-woven fabric layer;
an adhesive layer having an upper surface and a corresponding lower surface, the upper surface of the adhesive layer being attached to the bottom surface of the non-woven fabric layer, wherein the adhesive layer is configured to facilitate removably attaching the non-woven fabric layer to the portion of the user's upper and lower lips;
a plurality of spaced apart apertures formed to extent through the adhesive layer to provide air flow between the upper surface and the lower surface of the adhesive layer;
a removable backing being configured to cover the non-woven fabric layer and the adhesive layer, wherein the removable backing is removably attached to the lower surface of the adhesive layer;
wherein the non-woven fabric layer has a perimeter and includes a flexible member encasing the perimeter; and
wherein the adhesive layer has an identical width and length of the non-woven fabric layer.

16. The anti-snoring device according to claim 15, wherein the non-woven fabric layer is formed from viscose rayon.

17. The anti-snoring device according to claim 15, wherein the adhesive layer is formed from acrylate copolymer.

18. The anti-snoring device according to claim 15, wherein the removable backing is one or more of: a cotton backing, a biodegradable plastic material backing, a paper backing.

19. The anti-snoring device to claim 15, wherein a laminate is attached to the top surface of the non-woven fabric layer, the laminate comprising a breathable film.

20. The anti-snoring device according to claim 15, wherein the removable backing has an identical width and length of the non-woven fabric layer.
